(19) United States
(12) Patent Application Publication
(10) Pub. No.: US 2008/0021550 A1
(43) Pub. Date: Jan. 24, 2008

(54) Accommodative intraocular lens having a single optical element

(76) Inventor: Gary A. Richardson, Rochester, NY (US)

Correspondence Address: Bausch & Lomb Incorporated, One Bausch & Lomb Place, Rochester, NY, 14604-2701, US

(21) Appl. No.: 11/489,063

(22) Filed: Jul. 19, 2006

(51) Int. Cl. A61F 2/16

(52) U.S. Cl. 623/6.37; 623/6.51; 623/6.44

(57) ABSTRACT

An accommodative intraocular lens (AIOL) adapted to fit in a capsular bag, having an optic; at least three haptic arms, each arm being coupled to the optic along the arm's length and at least three plates, each plate having an outer surface arranged to contact the capsular bag and each plate being coupled to at least two of the haptic arms, the coupling with each arm occurring at a connection. The haptic arms and plates, in combination, may be arranged to form a closed figure surrounding the optic. A first area of the outer surface of at least one of the plates may be disposed anteriorly of a centroid of the connection with at least one of the plates, and a second area of the outer surface of the at least one of the plates being disposed posteriorly of the centroid, the first area and the second area being within 200% of one another in magnitude.

### FIELD OF INVENTION

The present invention relates to accommodative intraocular lenses, and more particularly to accommodative intraocular lenses having a single optical element.

### BACKGROUND OF THE INVENTION

FIG. 1 illustrates a cross-sectional view of a healthy human eye 110 having an anterior chamber 112 and a posterior chamber 114 separated by an iris 130. Within the posterior chamber 114 is a capsular bag 116 which holds the eye's natural crystalline lens 117. The capsular bag has an equatorial region 117.

Light enters the eye by passing through cornea 118. The cornea and crystalline lens act together to direct and focus the light onto retina 120. The retina is connected to optic nerve 122 which transmits images received by the retina to the brain for interpretation. Eye 110 has a visual axis VA.

In response to the sharpness of the image received by the retina, the brain operates to contract or relax ciliary muscle 126. Ciliary muscle 126 is disposed within ciliary body 128, and upon contraction of the ciliary muscle, the ciliary body is caused to move. To achieve near focus accommodation, the ciliary muscle is contracted thereby causing the ciliary body to relax tension on zonules 127 which permits the capsular bag and lens 117 to become more rounded. To achieve far focus (i.e., disaccommodation), the ciliary muscle is relaxed thereby increasing tension on zonules 127 which causes the capsular bag and lens 117 to become flatter.

In an eye where the natural crystalline lens has been damaged (e.g., clouded by cataracts), the natural lens is no longer able to properly focus and/or direct incoming light to the retina. As a result images become blurred. A well known surgical technique to remedy this situation involves removal of a damaged crystalline lens through a hole in the capsular bag known as a capsularhexis (also referred to simply as a rhexis). Subsequently, an artificial lens known as an intraocular lens (IOL) can be placed into the evacuated capsular bag through the rhexis.

Conventional IOLs are typically fixed-focus lenses. Such lenses are usually selected to have a power such that the patient has a fixed focus for distance vision, and the patient requires spectacles or contact lenses to permit near vision. In recent years extensive research has been carried out to develop IOLs having variable focus capability. Such IOLs are known as accommodating IOLs (AIOLS). The term “AIOLs” refers to both single-element and multi-element lenses.

AIOLs permit a wearer to have accommodative vision. AIOLs are typically located in the posterior chamber (e.g., in the capsular bag) and provide variable focal power in accordance with tension or a lack of tension exerted on the capsular bag 116 as a result of contraction and relaxation of the ciliary muscle.

One example of a single-element AIOL is given in PCT Applications WO 2006/040041, by Humanoptics, filed Oct. 8, 2006. The substance of said application is hereby incorporated by reference. Relevant portions of FIG. 6 of said application are reproduced herein as FIG. 2. FIG. 2 illustrates an AIOL 208 comprising an optic 201 (also commonly referred to as an optical element), three haptic arms 202a, 202b and 202c (collectively referred to as haptics arms 202) and three plates 204a, 204b and 204c (also commonly referred to as footplates). Openings 227 are formed between optic 201, plates 204a, 204b, 204c and arms 202a, 202b and 202c. FIG. 3 illustrates a cross sectional side view of AIOL 208 taken along line III-III of FIG. 2. Arrow 211 extends in an anterior direction and arrow 214 extends in a posterior direction. The intended operation of AIOL 208 occurs by optic 201 moving anteriorly in response to pressure from a patient's capsular bag, in response to relaxation of the ciliary muscle. To date, to the best of the present Applicant's knowledge, the lens illustrated in FIG. 6 has provided insufficient accommodation.

### SUMMARY

Aspects of the present invention arise from a recognition by the inventor (using computer simulation) that a drawback exists with prior art, single optical element, lenses as described with reference to FIG. 2 above. In particular, it was recognized that the portion of a plate outer surface that is disposed anteriorly of the connection of the plate with a corresponding haptic arm is too large relative to the portion of the plate that is disposed posteriorly to the connection. As a result, the inventor determined that there is a substantial predisposition of the lens to resist anterior (i.e., accommodative) motion of the lens, thereby limiting the accommodative movement that is achieved.

As described in greater detail below, aspects of the present invention are directed to the configuration and arrangement of the plates of lenses having single element optics. In particular, in lenses according to aspects of the present invention, the posterior portion of the plate outer surface and the anterior portion of the plate outer surfaces are more equally distributed than in prior art lenses.

A first aspect of the invention is directed to an accommodative intraocular lens (AIOL) adapted to fit in a capsular bag, comprising an optic, at least three haptic arms, each arm being coupled to the optic along the arm's length, at least three plates, each plate having an outer surface arranged to contact the capsular bag and each plate being coupled to at least two of the haptic arms, the coupling with each arm occurring at a connection. The haptic arms and plates, in combination, are arranged to form a closed figure surrounding the optic. A first area of the outer surface of at least one of the plates is disposed anteriorly of a centroid of the connection with at least one of the arms, and a second area of the outer surface of the at least one of the plates is disposed posteriorly of the centroid. The first area and the second area are within 200% of one another in magnitude.

Another aspect of the invention is directed to an accommodative intraocular lens adapted to fit in a capsular bag, comprising an optic having an optical axis, at least three haptic arms, each arm being coupled to the optic along the arm's length, at least three plates, each plate having an outer surface arranged to contact the capsular bag and each plate being coupled to at least two of the haptic arms, the coupling with each arm occurring at a connection. The haptic arms and plates, in combination, are arranged to form a closed figure surrounding the optic, and midpoint of the outer surface of at least one of said plates in the direction of the optical axis, and a midpoint of the connection in the direction of the optical axis being substantially on a common plane that is perpendicular to the optical axis.

Yet another aspect of the invention is directed to an accommodative intraocular lens adapted to fit in a capsular bag, comprising an optic, at least three haptic arms, each arm being coupled to the optic along the arm's length, and at least three plates, each plate having an outer surface arranged to contact the capsular bag and each plate being coupled to at least two of the haptic arms, the coupling with each arm occurring at a connection. The haptic arms and plates, in combination, are arranged to form a closed figure surrounding the optic. The plate are configured and arranged such that there is substantially no moment of a haptic arm that would produce posterior translation of the optic, when the optic is placed in the capsular bag.

### BRIEF DESCRIPTION OF THE DRAWINGS

Illustrative, non-limiting embodiments of the present invention will be described by way of example with reference to the accompanying drawings, in which a same reference number is used to designate a same or similar components in different figures, and in which:

FIG. 1 illustrates a cross-sectional view of a human eye;

FIG. 2 is a top view (anterior side) of a prior art AIOL;

FIG. 3 is a cross sectional side view taken along plane III-III of FIG. 2;

FIG. 4A is a top view (anterior side) of a first embodiment of an lens according aspects of the invention;

FIG. 4B is a schematic view of a connection between a haptic arm and a plate taken along line IV-IV of FIG. 4A and showing a portion of a plate in the background;

FIG. 5 is a cross sectional view taken along plane V-V of FIG. 4A;

FIG. 6 is a top view of the anterior side of a second embodiment of the lens according to aspects of the invention;

FIG. 7 is a cross sectional side view of the lens taken along line VII-VII of FIG. 6;

FIG. 8A a top view of the front side of the third embodiment of an lens according to aspects of the invention;

FIG. 8B is a schematic view of a connection between a haptic arm and a plate taken along line VIII-VIII of FIG. 8A and showing a portion of a plate in the background; and

FIG. 9 is a cross sectional view taken along plane IX-IX of FIG. 8A.

### DETAILED DESCRIPTION

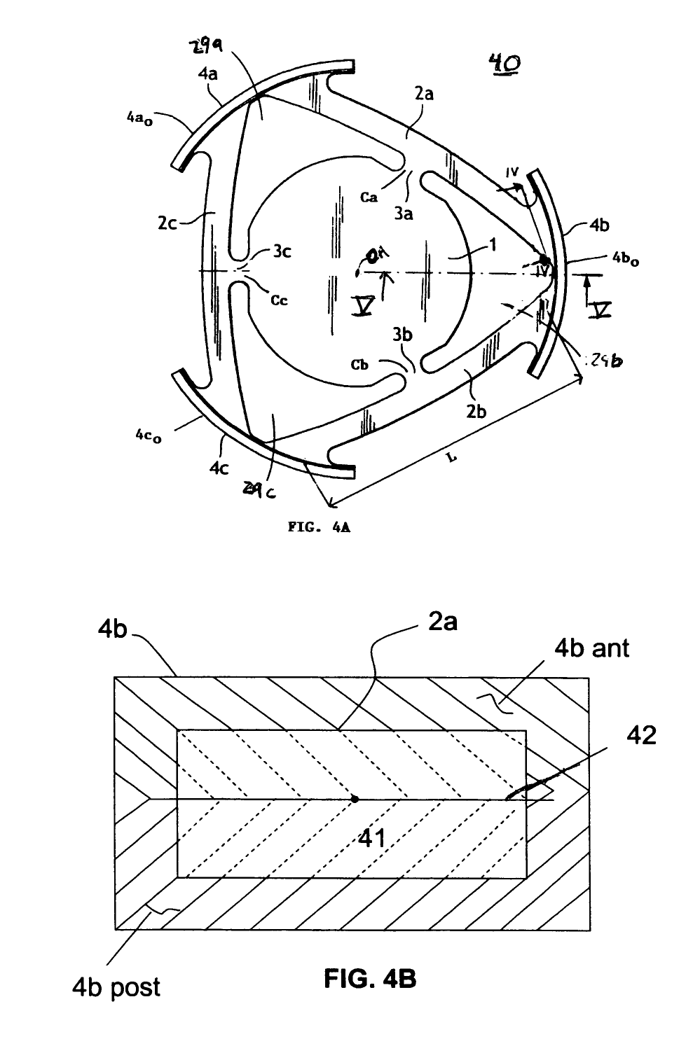

FIG. 4A illustrates an accommodative intraocular lens (AIOL) 40 adapted to fit in a capsular bag of an eye. Lens 40 comprises an optic 1, three haptic arms 2a, 2b and 2c, and three plates 4a, 4b and 4c.

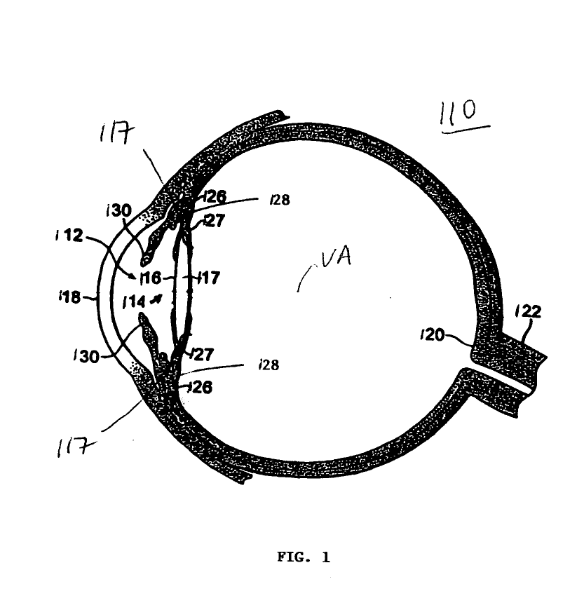

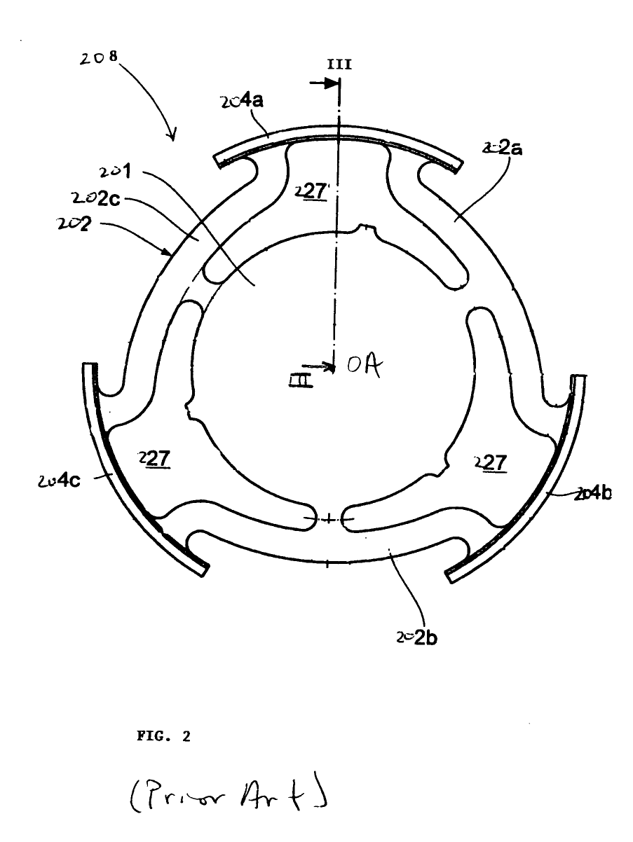

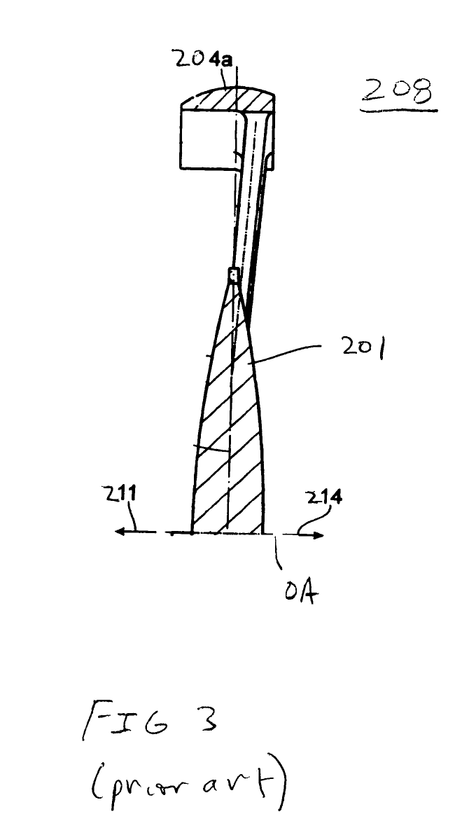

The haptic arms may be arcuate when viewed from the anterior side (as illustrated in FIG. 1), linear or have a more complex shape. The arms will have a generally elongate shape, with a length L. Each arm is coupled to the optic along its length L at a corresponding connection Ca, Cb, Cc. In some embodiments, the coupling occurs substantially at a point midway along the length of a given haptic. In the illustrated embodiment, arms 2a, 2b, 2c are each coupled to optic 1 via a connective segment 3a, 3b, 3c. However, any suitable connection technique may be used.

In FIG. 4A, the arms 2a, 2b, 2c substantially form an equilateral triangle, the sides of which are slightly deformed and are outwardly convex from the optic. The arms are connected with three plates 4a, 4b, 4c at the vertices of the triangle. Each plate is coupled to at least two of the haptic arms. The haptic arms and plates, in combination, are arranged to form a closed figure (i.e., a triangle) surrounding the optic. In some such embodiments, the arms by themselves may form a closed figure, with the haptics being connected to the arms at vertices of the closed figure. In some embodiments of IOLs having three haptic arms, the haptic arms (perhaps in combination with the plates) may form a circle due to their arcuate shapes. Openings 29a, 29b, 29c are located between adjacent haptic arms and optic 1. In some embodiments, each opening 29 is partially bounded by a plate, the optic (possibly a frame of an optic) and at least two haptics arms. In other embodiments, the openings are bounded by the optic and haptics only. Each plate 4a, 4b, 4c has an outer surface 4a_{O}, 4b_{O}, 4c_{O} arranged to contact the capsular bag of an eye.

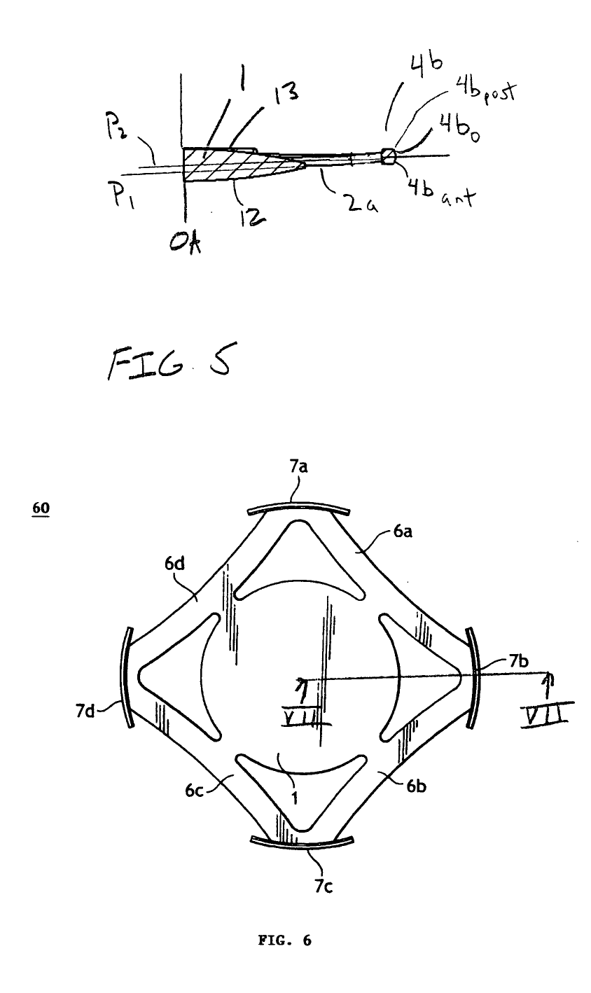

FIG. 5 is a cross sectional side view taken along line V-V of FIG. 4A. Optic 1 is illustrated as biconvex. However, the optic surfaces can have any suitable shape (e.g., convex or concave), and can be spherical or aspheric. FIG. 5 illustrates arm 2b as being arched, such that the portion of the haptic arm that are connected to optic 1 are located posteriorly to the portion of the haptic arms that is connected to plate 4b. The vertices of the triangle (shown in FIG. 4a) and the connection of the arms with the plates (shown in FIG. 4a) are disposed on plane P2. A middle plane P1 of the optic 1 lies slightly in front of plane P2. Optic 1 has an anterior surface 12 and a posterior surface 13, which lies opposite to the anterior surface 12.

Referring again to FIG. 4A, in some embodiments, the outer surface of the plates (e.g., plate 4b_{O}) form a substantially toric surface to contact the equatorial region of the capsule sac. It is to be appreciated that the plates and arms are configured and arranged such that applying a force on the outer surfaces of the plates (i.e., in a manner in which a force would be applied by the inner surface of a capsular bag in response to contraction of the ciliary muscle) affects a shift of optic 1 in the anterior direction.

According to some aspects of the invention, a first area 4b_{ant.} of the outer surface of at least one of the plates is disposed anteriorly of a centroid of the connection with the at least one plate and at least one of the arms; and a second area 4bₚₒₛₜ of the outer surface of the at least one of the plates is disposed posteriorly of the centroid. The first area and the second area are perferably within 200% of one another in magnitude (i.e., the area constituting the first area is less than two-times larger than the area constituting the second area, and the area constituting the second area is less than two-times the area constituting the first area). In some embodiments, the first and second areas are within 150% of one another. In other embodiments, the first and second areas are within 125% of one another.

FIG. 4B is a cross sectional view of a connection between a haptic arm and a plate taken along line IV-IV of FIG. 4A. It will be appreciated that the connection has an area along the cross section, which has a centroid 41. Centroid 41 is disposed in the plane of the cross section. Centroid 41 is also disposed on a line 42 that is perpendicular to the direction of the optical axis OA (shown in FIG. 5). Referring again to FIG. 5, first area 4bₐₙₜ lies on the anterior side of a plane that includes line 42 and is perpendicular to the optical axis; and second area 4bₚₒₛₜ lies on the posterior side of the plane extending through line 42 and perpendicular to the optical axis. Both 4bₐₙₜ and 4bₚₒₛₜ include portions obscured by the connection. (These obscured portions are illustrated in dashed line in FIG. 4B.) The first area and the second area are calculated over the entire plate width, including portions where another haptic arm is connected to the plate.

In some embodiments, a midpoint of outer surface 4a_{O} of at least one of said plates, and a midpoint of the connection are disposed substantially on a common plane that is perpendicular to the optical axis. A midpoint is the middle of a surface as measured along the direction of optical axis OA. It is to be appreciated that, while in the illustrated embodiment the midpoint is coincident with the centroid, in other embodiments they will not be coincident.

In some embodiments, at least one of the plates is configured and arranged such that there is substantially no moment of a haptic arm that would produce posterior translation of the optic in response to a force applied to the outer surface of the haptic. In some of the embodiments, all of the haptics are so configured. As discussed above, a problem with the prior lenses is the existence of moment that causes a translation force in a posterior direction when the lens is placed in the eye. Such a force resists accommodative (i.e., anterior) movement of the lens. While in some embodiments of the present invention provide no moment to the optic when force is applied to the outer surfaces of the plates by a capsular bag, in other embodiments, the moment is substantially reduced from the prior, but is not completely absent.

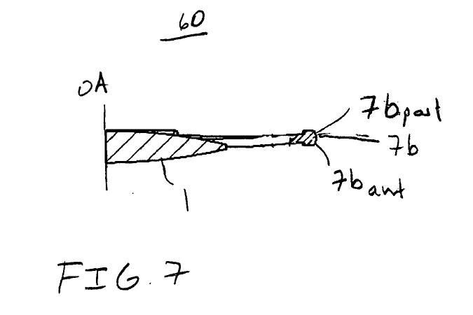

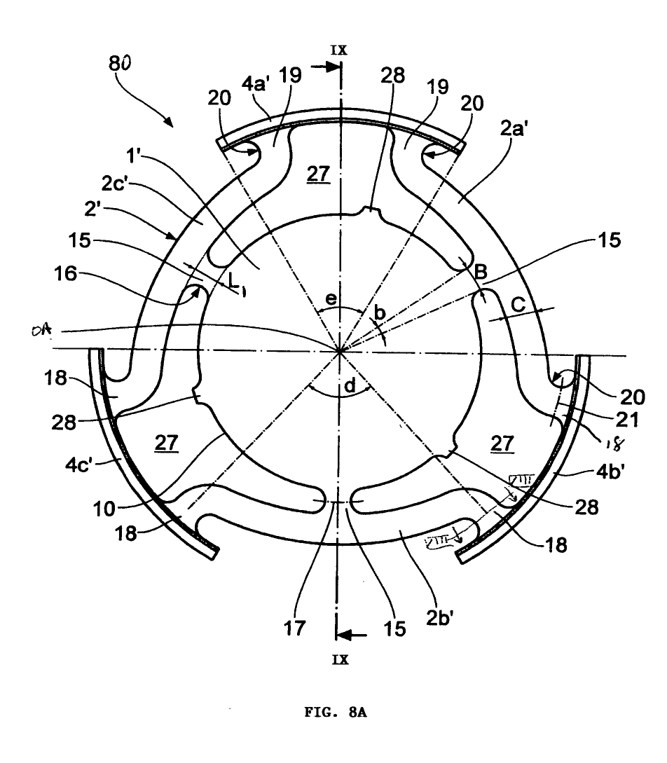

FIGS. 6 and 7 illustrate another embodiment of an AIOL 60 according to aspects of the invention. AIOL 60 comprises four inwardly convex arms 6a, 6b, 6c, 6d, which form a quadrilateral. Plates 7a, 7b, 7c, and 7d disposed at the vertices of the quadrilateral. It is to be appreciated that, similar to the embodiment discussed with reference to FIG. 4A, the plates and arms are configured and arranged such that applying a force on the outer surfaces of plates 7a, 7b, 7c, and 7d (i.e., in a manner in which a force would be applied by the inner surface of a capsular bag) affects an anterior shift of optic 1.

Also, similar to the lens discussed above with reference to FIGS. 4A and 5, AIOL 60 is configured such that a first area 7b_{ant.} of the outer surface of at least one of the plates is disposed anteriorly of a centroid of a connection with at least one of the arms; and a second area 7bₚₒₛₜ of the outer surface of the at least one of the plates is disposed posteriorly of the centroid. The first area and the second area is perferably within 200% of one another in magnitude. In some embodiments, the plates are configured such that the anterior portion of each plate has the same magnitude as the anterior portions each of the other plates; and the posterior portion of each plate has the same magnitude as the posterior portion of each of the other plates.

In some embodiments, a midpoint of the outer surface of at least one of said plates, and a midpoint of the connection are disposed substantially on a common plane that is perpendicular to the optical axis. In some embodiments, at least one of the plates is configured and arranged such that there is substantially no moment of a haptic arm that would produce posterior translation of the optic.

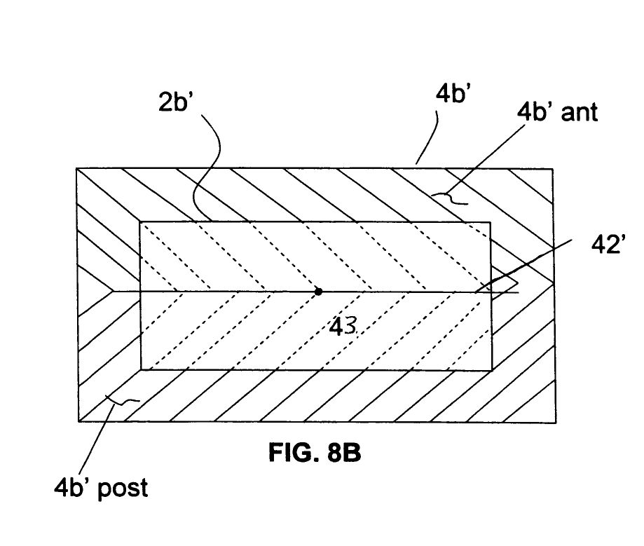

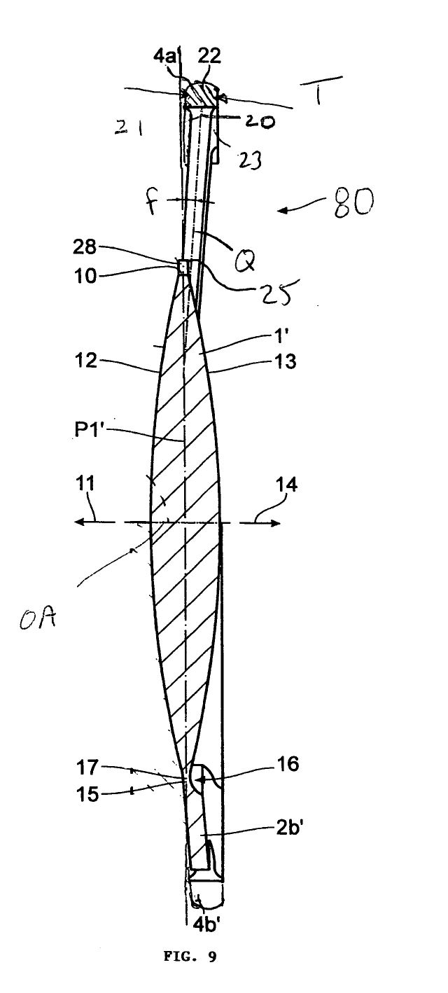

FIGS. 8A and 9 illustrate another embodiment of a lens 80 according to aspects of the invention. The optic 1′ has an optical axis OA, as well as a middle plane P1′ that is perpendicular to optical axis OA and runs through the center of the optic. The optic may be provided with a sharp external edge 25 about its periphery which serves as a barrier to cell migration.

Three tab-shaped connective segments 15 that are disposed 120° from each other are arranged and protrude outward in the edge 10 region of the optic 1′. The connective segments 15 are comparatively narrow and exhibit a width B that corresponds to a central angle b of approximately 5°≦b≦20°. In some embodiments b=10′.

In the illustrated embodiment, each connective segment 15 comprises a hinge 16. Hinges 16 exhibit corresponding pivot axes 17 which is disposed substantially in the center of the hinge 16 and are disposed substantially along middle plane P1′. On the posterior side of the lens, the connective segments 15 each include a groove-shaped depression that forms the hinge. The connective segments 15 exhibit a radial length L1 that in some embodiments is equal to approximately the width B.

Haptic arms 2a′, 2b′, and 2c′ are disposed at the outer ends of the connective segments 15. Each arm is coupled to the optic along its length L′. The arms 2a′, 2b′, and 2c′ are convexly arched about the optical axis OA, and in their middle section run in an approximately circular arc, such that the distance between the edge 10 and the arms 2a′, 2b′, and 2c′ is approximately constant. In the illustrated embodiment, the arms 2a′, 2b′, and 2c′ exhibit a width C that is approximately equal to width B. Both outer ends 18 of each arm 2a′, 2b′, and 2c′ are connected with a plate 4a′, 4b′, and 4c′. In some embodiments, ends 18 belonging to a given one of the arms 2a′, 2b′, and 2c′ subtend an angle d with reference to the axis OA of about 70°≦d≦105°. In the illustrated embodiment d≈90°.

Each plate has an outer surface arranged to contact the capsular bag. Each plate is coupled to at least two of the haptic arms, the coupling with each arm occurring at a connection C_{d}, Cₑ, and C_{f}. For example, the adjacent ends 18 of arms 2b′ and 2c′ are connected with plate 4c′. The arms 2a′, 2b′, and 2c′ and the plates 4a′, 4b′, and 4c′ roughly comprise an equilateral triangle in which the vertices of the triangle are formed by the plates 4a′, 4b′, and 4c′. It will be appreciated that, in some embodiments, due to the arcuate shape of the haptic arms and plates the arms and plates may have a substantially circular shape. The haptic arms and plates, in combination, being arranged to form a closed figure surrounding the optic. Plates 4a′, 4b′, and 4c′ each exhibit a central angle e with reference to optical axis OA. In some embodiments, 45°≦e≦75°. In the illustrated embodiments, e≈60°.

Plates 4a′, 4b′, and 4c′ extend in an angular direction over the ends 18 of each of the arms. An arm 2a′, 2b′, and 2c′, which comprises a largely tangentially running portion and protruding connective segment 19. The connective segment extends in a radial direction in the region of the ends 18 of the arms. The thickness T of the plates is typically 0.3-0.7 mm.

In some embodiments, the connective segment at which the haptic arms couple to the plates each include a hinge 20 that exhibits a pivot axis 21 which is parallel to the middle plane P1′ and displaced from the plane. Hinge 20 enables relative pivoting of an arm and corresponding plate. Although the present embodiment is described as having hinges 16 and 20, it is possible to omit one or both of the hinges on a given arm. In embodiments in which both hinges are omitted, each arm 2a′, 2b′, or 2c′ may have a largely constant thickness to transition into the plate 4a′, 4b′, or 4c′ (i.e., the haptic arms are formed without either one or both of locally defined external hinge 20 or 16). In such embodiments, the segment of each arm between the connective segment 15 and each plate 4a′, 4b′, or 4c′ assumes the function of the hinges 16 and 20. This result is achieved by selecting an arm thickness that provides continuous deformation along the length of the arm in response to a radial force applied to the outer surface of a corresponding plate. Accommodative movement is thereby achieved.

Each plate 4a′, 4b′, and 4c′ exhibits a cylindrical interior surface 23, which faces toward the axis OA. The plates 4a′, 4b′, and 4c′ exhibit a toroidal exterior surface 22. The connective segments 15, the arms 2a′, 2b′, and 2c′, and the plates 4a′, 4b′, and 4c′ together comprise the haptic 2′, which supports the optic 1′ against the equatorial region of the capsular sac. Typically, the plates extend at least partially beyond the haptic arm in both the anterior and the posterior directions. However, the invention is not so limited.

In an unstressed condition, plates 4a′, 4b′, and 4c′ are disposed posteriorly of middle plane P1′. Angle f is defined as the angle between the middle plane P1′ and a line Q through the middle of hinges 16 and the middle of hinges 20, where 2°≦f≦25°, and preferably f≈5° and corresponds, in other words, to a slight biasing of the lens 80 in the anterior direction.

Similar to the lens discussed above with reference to FIGS. 4A and 5, lens 1′ is configured such that a first area 4b_{ant.}′ of the outer surface of at least one of the plates is disposed anteriorly of a centroid of the connection with at least one of the arms; and a second area 4bₚₒₛₜ′ of the outer surface of the at least one of the plates is disposed posteriorly of the centroid. The first area and the second area are perferably within 200% of one another in magnitude. As was described above, in some embodiments, the first and second areas are within 150% of one another. In other embodiments, the first and second areas are within 125% of one another. Typically, the haptic arms and plates of a given AIOL are identically configured.

FIG. 8B is a cross sectional view of a connection between a haptic arm and a plate taken along line VIII-VIII of FIG. 8A. It will be appreciated that the connection has an area along the cross section, which has a centroid 43. Centroid 43 is disposed in the plane of the cross section. Centroid 43 is also disposed on a line 42′ that is perpendicular to the direction of the optical axis OA (shown in FIG. 9). First area 4bₐₙₜ′ lies on the anterior side of a plane that includes line 42 and is perpendicular to the optical axis; and second area 4bₚₒₛₜ′ lies on the posterior side of the plane extending through line 42′ and perpendicular to the optical axis. Both 4bₐₙₜ′ and 4bₚₒₛₜ′ include portions obscured by the connection. (These portions are illustrated in dashed line.) In some embodiments, a midpoint of the area of the outer surface of at least one of said plates, and a midpoint-of the connection are disposed substantially on a common plane that is perpendicular to the optical axis. In some embodiments, the plate is configured and arranged such that there is substantially no moment of a haptic arm that would produce posterior translation of the optic. While in some embodiments there is substantially no moment, in other embodiments of the invention, there remains a moment, but the moment is substantially reduced from that of the prior art lenses.

Three openings 27 are defined by two adjacent arms (e.g., 2a′ and 2c′). In some embodiments, a part of edge 10 of optic 1′, as well as the middle section of the interior surface 23 of a plate such as the plate 4a′. A protrusion 28 that is radially off-center may be provided in the region of its edge 10. The protrusions enable the recognition an anterior side and the posterior side of the lens 80. If the lens 80 is placed correctly in the capsular sac, each protrusion 28 in the corresponding opening 27 will be located in the right half of the opening 27 from the perspective of the physician. If the placement is in the incorrect orientation, each protrusion 28 will be located in the left half of the corresponding opening 27.

Lens 80 may, instead of three plates, contain a larger number, for example, four, five, six, seven or more plates with a corresponding number of arms. As described above, in each case, the arms and plates, in combination, are arranged to form a closed figure surrounding an optic.

The implantation and accommodative behavior of the lens 80 will now be described in brief. After placing the circular capsulorhexis and removing the natural lens from the capsular sac in the eye of a patient, the lens 80 is introduced into the capsular sac through a small slit in the cornea so that the anterior direction 11 of the lens 80 is oriented toward the front of the eye, and the posterior direction 14 is oriented toward the rear in the direction of the retina. The optical axis OA of the lens and visual axis VA of the eye are substantially aligned.

The plates 4a′, 4b′, and 4c′ are positioned to bear against the inside of the capsular sac in its equatorial region. The optic 1′ is arched in the anterior direction 11 in the sense that the angulation angle f is greater than 0° so that the exterior pivot axes 21 lie behind the pivot axes 17. This state corresponds to distance vision in the human eye. Upon contraction of the patient's ciliary muscles a radial force is exerted inward on the plates 4a′, 4b′, and 4c′. This causes a displacement of the optic 1′ in the anterior direction 11, i.e., the lens is more pronouncedly biased toward the front. Such displacement provides near vision for the patient. In the accommodative process, the optic 1′ remains largely unchanged, such that its optical properties remain unchanged and accommodation is achieved through displacement of the lens.

It will be appreciated that, while implantation and accommodation was discussed with reference to lens 80, the implantation and accommodation of lenses 40 and 60 (discussed above) will be substantially similar.

Lenses according to aspects of the present invention are preferably made from materials that are reversibly deformable. This means that after deformation, e.g., by pressure exerted on the haptics, the material returns to its original state once the external pressure is removed. For example, lenses may be manufactured of transparent silicone or acrylic. In some instances, pHEMA, poly-hydroxyethyl-methacrylate is used.

The lens may be produced, for example, by molding or machining or a combination of both. In some instances in which the lens is machined, the material is preferably kept in a rigid state during processing. The product resulting from the machining is then placed in an aqueous saline solution, whereby the lens absorbs water and becomes elastic. Lenses may be manufactured as a single integrated unit with the lens including an optic, haptics and plates; however, the various parts may be individually manufactured and subsequently assembled.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An accommodative intraocular lens (AIOL) adapted to fit in a capsular bag, comprising:
an optic;
at least three haptic arms, each arm being coupled to the optic along the arm's length;
at least three plates, each plate having an outer surface arranged to contact the capsular bag and each plate being coupled to at least two of the haptic arms, the coupling with each arm occurring at a connection,
the haptic arms and plates, in combination, being arranged to form a closed figure surrounding the optic,
a first area of the outer surface of at least one of the plates being disposed anteriorly of a centroid of the connection with at least one of the arms, and a second area of the outer surface of the at least one of the plates being disposed posteriorly of the centroid, the first area and the second area being within 200% of one another in magnitude.

2. The AIOL of claim 1 wherein the haptic arms are linear.

3. The AIOL of claim 1 wherein the haptic arms are arcuate.

4. The AIOL of claim 3 wherein the haptic arms are outwardly convex with respect to an optical axis of the optic.

5. The AIOL of claim 3 wherein the haptic arms are inwardly convex with respect to an optical axis of the optic.

6. The AIOL of claim 1 wherein the haptic arms form a triangle.

7. The AIOL of claim 1 wherein the haptic arms form a quadrilateral.

8. The AIOL of claim 1 wherein the haptic arms form a circle.

9. The AIOL of claim 1 wherein the haptic arms form the closed figure by themselves.

10. The AIOL of claim 1 wherein each arm is coupled to the optic at a point midway along the length of the arm.

11. The AIOL of claim 1 wherein the first area and the second area are within 150% of one another in magnitude.

12. The AIOL of claim 1 wherein the first area and the second area are within 125% of one another in magnitude.

13. An accommodative intraocular lens adapted to fit in a capsular bag, comprising:
an optic having an optical axis;
at least three haptic arms, each arm being coupled to the optic along the arm's length;
at least three plates, each plate having an outer surface arranged to contact the capsular bag and each plate being coupled to at least two of the haptic arms, the coupling with each arm occurring at a connection,
the haptic arms and plates, in combination, being arranged to form a closed figure surrounding the optic,
a midpoint of the outer surface of at least one of said plates in the direction of the optical axis, and a midpoint of the connection in the direction of the optical axis being substantially on a common plane that is perpendicular to the optical axis.

14. The AIOL of claim 13 wherein the haptic arms are linear.

15. The AIOL of claim 13 wherein the haptic arms are arcuate.

16. The AIOL of claim 15 wherein the haptic arms are outwardly convex with respect to the optical axis.

17. The AIOL of claim 15 wherein the haptic arms are inwardly convex with respect to the optical axis.

18. The AIOL of claim 13 wherein the haptic arms form a triangle.

19. The AIOL of claim 13 wherein the haptic arms form a quadrilateral.

20. The AIOL of claim 13 wherein the haptic arms form a circle.

21. The AIOL of claim 13 wherein the haptic arms form the closed figure by themselves.

22. The AIOL of claim 13 wherein each arm is coupled to the optic at a point midway along the length of the arm.

23. An accommodative intraocular lens adapted to fit in a capsular bag, comprising:
an optic;
at least three haptic arms, each arm being coupled to the optic along the arm's length;
at least three plates, each plate having an outer surface arranged to contact the capsular bag and each plate being coupled to at least two of the haptic arms, the coupling with each arm occurring at a connection,
the haptic arms and plates, in combination, being arranged to form a closed figure surrounding the optic,
the plate being configured and arranged such that there is substantially no moment of a haptic arm that would produce posterior translation of the optic, when the optic is placed in the capsular bag.

24. The AIOL of claim 23 wherein the haptic arms are linear.

25. The AIOL of claim 23 wherein the haptic arms are arcuate.

26. The AIOL of claim 25 wherein the haptic arms are outwardly convex with respect to an optical axis of the optic.

27. The AIOL of claim 25 wherein the haptic arms are inwardly convex with respect to an optical axis of the optic.

28. The AIOL of claim 23 wherein the haptic arms form a triangle.

29. The AIOL of claim 23 wherein the haptic arms form a quadrilateral.

30. The AIOL of claim 23 wherein the haptic arms form a circle.

31. The AIOL of claim 23 wherein the haptic arms form the closed figure by themselves.

32. The AIOL of claim 23 wherein each arm is coupled to the optic at a point midway along the length of the arm.
